(19) United States
(12) Patent Application Publication
(10) Pub. No.: US 2019/0365952 A1
(43) Pub. Date: Dec. 5, 2019

(54) TISSUE SCAFFOLD MOLD APPARATUS AND USE IN MAKING TISSUE ENGINEERED ORGANS WITH HOLLOW STRUCTURES

(71) Applicant: THE JOHNS HOPKINS UNIVERSITY, Baltimore, MD (US)

(72) Inventors: Anirudha Singh, Baltimore, MD (US); Trinity Bivalacqua, Baltimore, MD (US); Nikolai Sopko, Baltimore, MD (US); David Lee, Baltimore, MD (US)

(21) Appl. No.: 16/478,190

(22) PCT Filed: Jan. 19, 2018
(86) PCT No.: PCT/US2018/014426

§ 371 Date: Jul. 16, 2019

Related U.S. Application Data
(60) Provisional application No. 62/448,973, filed on Jan. 21, 2017.

(51) Int. Cl. A61L 27/38; A61L 27/24; A61L 27/54; A61L 27/56; B29C 45/00

(52) U.S. Cl. CPC A61L 27/3882; A61L 27/24; A61L 27/54; A61L 27/3834; B29K 2105/0073; B29C 45/0001; A61L 2430/40; A61L 2430/22; A61L 27/56

(57) ABSTRACT

The present invention provides a tissue scaffold mold apparatus and methods for use of the mold apparatus to simply, rapidly and easily form molded tissue scaffolds from fibrous proteins such as collagen, and with other matrix components having complex 3-dimensional designs that can be seeded with stem cells for creating biologically and mechanically functional tissues/grafts. The inventive methods and apparatus allows for tissue engineering of hollow or concave and tubular organs and tissues, and will have immediate impact in a wide range of biomedical areas from tissue engineering, regeneration and reconstructive surgery.

### REFERENCE TO RELATED APPLICATIONS

This application claims the benefit of U.S. Provisional Patent Application No. 62/448,973, filed on Jan. 21, 2017, which is hereby incorporated by reference for all purposes as if fully set forth herein.

### BACKGROUND OF THE INVENTION

Bladder cancer is an adverse health condition affecting nearly 2.7 million people worldwide (World J. Urol. 27, 289-293 (2009)). It is estimated that in 2015, ˜74,000 patients were newly diagnosed and ˜16,000 patients died due to the urinary bladder cancer in USA (NCI-statistics. SEER Stat Fact Sheets: Bladder Cancer 2015). The current gold-standard surgical option available for patients with muscle invasive bladder cancer (MIBC) is complete bladder removal or “radical cystectomy (RC),” although other surgical options of bladder augmentation and replacement are also available in special conditions (FIG. 1) (J. Urol. 155, 2098-2104 (1996); J. Urol. 156, 571-577 (1996); J. Urol. 185, 562-567 (2011)). After RC, a urinary diversion is necessary and typically a conduit is created from segments of the healthy gastrointestinal tract, commonly the ileum; however, patients with an ileal conduit are prone to many health complications, including metabolic disturbances, stone formation, urine-leakage and chronic infections due to the inherent absorptive and secretory properties of gastrointestinal segments, as well as renal compromise with early development of chronic kidney disease (Adv. Urol., 2011, 764325 (2011); J. Urol. 147, 1199-1208 (1992); J. Urol. 193, 891-896 (2015); BJU international 108, 330-336, (2011); Current opin. Urol. 25, 570-577, (2015); J. Urol. 169, 985-990 (2003)).

Tissue-engineered (TE) urinary tissues (FIG. 1) for human use that can eliminate or mitigate these challenges are feasible; however, their clinical translations are critically limited and have largely failed due to either insufficient mechanical properties or inadequate functional biological responses, such as contractibility, lack of vascularization, and anti-fibrosis properties (J. Tissue Eng. Regen. Med. 7, 515-522 (2013); PloS one 10, e0118653 (2015); Current Urol. Rep. 16, 8, (2015); Exp Biol Med (Maywood) 239, 264-271 (2014). For example, a recently completed clinical trial using PLGA electrospun scaffold with autologous urothelial and smooth muscle cells (Tengion™) failed to function, although the muscle and urothelial layers were histologically present. Furthermore, insufficient supply of healthy cells from the bladder tissues of these patients create a major challenge for their clinical translations; therefore, a relatively easily extractable human adipose-derived stem cells (hADSCs) are pivotal for creating a successful TE conduit. Passively seeding cells on a biodegradable scaffold that may potentially replicate biological functions of these tubular and hollow organs has not been shown to be optimal and thus incomplete (J. Urol. 191, 1389-1395 (2014)). For TE to tangibly advance regenerative urology, it is essential to develop noble yet comprehensive approaches that are scientifically sound, broadly applicable and commercially viable.

Urological tissues are collagen-based hollow and tubular structures consisting of urothelial lined mucosa, epithelial sub-mucosa in the lumen and orthogonally arranged surrounding muscle layers (Methods 47, 109-115 (2009)). As a structural protein, collagen not only modulates vital biological functions but also provides mechanical strength, physical support and shape to the tissues, critical for their physiological urodynamic functions, which makes collagen a natural choice for biomaterial applications (Materials 3, 1863-1887, (2010); Med Biol Eng Comput 38, 211-218, (2000)). However, TE collagen scaffolds have inherent challenges related to application-specific optimal mechanical and structural properties although mostly due to constraints in the molding methodology used in lab set up; and therefore its application is limited to soft tissue and non-load bearing applications (Science 215, 174-176 (1982); Biofabrication 7, 035005, (2015)). Previously, researchers have rolled collagen sheets, sutured or glued the ends (structurally weak points), and poured collagen solution into a mold to develop a tubular structure however, no attempts have been made to develop continuous and seamless designer collagen structures that can capture the urodynamic design of the ureter (Adv. Funct. Materials 15, 1762-1770 (2005); J. Tissue Eng. Regen. Med. 4, 123-130, (2010); Tissue Eng. Part A 21, 2334-2345, (2015); Biomaterials 33, 7447-7455 (2007)).

As such, there still exists and unmet need for developing methods for making seamless collagen structures that can replicate the 3-dimensional design of diseased or non-functional organs, such as the bladder and/or ureter, and provide a mechanically robust and functional collagen-based organs.

### SUMMARY OF THE INVENTION

In accordance with some embodiments, the present invention provides a newly developed biofabrication apparatus and process that leads to molded tissue scaffolds with unprecedented design features and user-controlled properties, which can create a mechanically robust and biologically functional urinary conduit. The inventive process resembles the features of polymer processing methods-vacuum thermoforming and stretch blow molding that shape synthetic polymers into desired structures and articles.

Specifically, the inventors demonstrate development of molded tissue engineered scaffolds, using collagen and the inventive apparatus, ranging from microureters to minibladders that are mechanically tunable and robust and can incorporate variable designs in longitudinal and transverse planes. It is anticipated that the inventive apparatus and methodology will have major scientific and clinical impact, and provides the foundation for constructing and regenerating hollow tissues, such as urological tissues.

In accordance with an embodiment, the present invention provides a mold apparatus for making a molded tissue scaffold comprising an inlet/outlet adaptor, wherein said inlet/outlet adaptor comprises an inlet port and an outlet port which can allow fluids and gases to pass through the inlet or outlet port of the inlet/outlet adaptor, said adaptor further comprising an internal mold element comprised of a sintered material which is semi-permeable or porous material or 3D printed material and said internal mold element defining a hollow interior space which connects to the outlet port of the adaptor and communicates with the outlet port of the inlet/outlet adaptor, said internal mold element is capable of allowing gas and fluid to pass through the exterior of the internal mold element into the hollow interior space of the internal mold element and out of the outlet port of the inlet/outlet adaptor; the mold apparatus further comprises a mold chamber which is comprised of at least one wall comprising a flexible material which defines the inside and outside of the mold chamber, and encloses the internal mold element, and which is fastened at one end, to the inlet/outlet adaptor; the inlet port of the inlet/outlet adaptor communicates with the interior of the mold chamber such that fluid and/or a liquid tissue composition can enter into the mold chamber and be contained within said chamber, and the liquid tissue composition can be added to the chamber at sufficient pressure to expand the flexible wall of the mold chamber such that the wall of the mold chamber will provide counter pressure to the liquid in the chamber and press the liquid against the internal mold element.

In accordance with an embodiment, the present invention provides a method for making a molded tissue scaffold comprising the steps of: a) solubilizing a solution comprising one or more fibrous proteins suitable for use as a tissue scaffold; b) combining the solution of a) with at least a second solution which will promote fibrogenesis of the protein solution of a); c) adding the combined solution of b) into the inlet of a mold apparatus capable of containing the solution of b) under pressure and gravity, and which comprises an internal mold element which is semi-permeable or porous and communicates at least one end to the outlet of the mold apparatus; d) condensing the solution of b) in the expandable mold chamber of the mold of c) via application of vacuum to the outlet of the mold and/or pressure from the expandable mold chamber, and removing water from the solution of b) until the scaffold has desired thickness and tensile strength; and e) removal of the molded tissue scaffold from the mold.

In accordance with an embodiment, the present invention provides a molded tissue scaffold comprising one or more fibrous proteins having the 3-dimensional shape of an organ of the body.

In accordance with another embodiment the present invention provides a molded tissue scaffold in a shape selected from the group consisting of: a ureter, bladder, urethra, small intestine, and a blood vessel.

In accordance with a further embodiment, the present invention provides a molded tissue scaffold described herein, for use in replacement of an organ in a body of a subject in need thereof.

In accordance with still another embodiment, the present invention provides a molded tissue scaffold described herein for use in the augmentation or supplementation of an organ in a body of a subject in need thereof.

### BRIEF DESCRIPTION OF THE DRAWINGS

FIGS. 1A-1D illustrate the human urinary system, and surgical & reconstructive approaches. 1A) Ureter carries filtered urine from kidney to a bladder, which acts as a reservoir that can be voided through urethra. Ureter and urethras are tubular, while bladder is a dome-shaped hollow tissue structure comprised of multiple cellular layers, including smooth muscle cell and urothelial cells, surrounded by collagen and elastin as the major components of extracellular matrices. On failure of urinary bladder due to cancer or any other medical conditions that require bladder removal, three surgical approaches are commonly practiced to create a new way to bypass urine outside of the body. 1B) a urinary diversion conduit constructed of ileum, 1C) partial bladder replacement or bladder augmentation and 1D) completely reconstructed neo bladder, constructed of gastrointestinal segments.

FIGS. 2A-2B illustrate a tubular embodiment of the mold apparatus of the present invention. 2A depicts the mold apparatus (10) with the mold chamber (16) unfilled, and 2B depicts the mold chamber (16) filled with a solubilized tissue scaffold solution in process of condensing the solution and allowing fibrogenesis and vitrification to occur by removing water from the solution via escaping through the internal mold element (14) and exiting via the outlet (13).

FIGS. 3A-3C depict both prior art vacuum thermoforming and blow-molding, and an embodiment of the collagen molding methods of the present invention. 3A) In a conventional vacuum thermoforming process, a soft heated plastic sheet is vacuum-pulled to stretch and form the desired shape that on cooling retains the features of the mold. 3B) In a stretch-blow molding process, a soft plastic tube is air-blown from inside to stretch and press against the wall of the mold or chamber forming the desired hollow structure, which on cooling retains the shape (for example, a plastic bottle). 3C) In an embodiment of the present invention, after addition of a solubilized tissue scaffold solution into the mold apparatus, after initiation of fibrogenesis, the wall of the mold chamber presses the tissue scaffold solution against the mold by applying vacuum, tissue scaffold articles (such as collagen) are formed onto removable or sacrificial semi-permeable or porous internal mold elements with predefined structures.

FIGS. 4A-4I illustrate designer tubular and hollow/bulbar or concave collagen articles for urological applications. Collagen can be molded into 4A) tubular structures as well as 4B) partial or full hollow or concave structures by applying a negative pressure or partial vacuum in a mold chamber with an internal mold element. A laboratory set up developed for the embodiment of 4C) a ureter-like tubular, and 4D) a bladder-like bulbous scaffold design. 4E) Tubes with various wall thicknesses corresponding to the initial volume load are added with Platelet-rich plasma (PRP) as an example for further enhancing the biological properties of scaffolds of some embodiments of the present invention. 4F) Loading 5-10 wt % PRP in tubes did not change the mechanical properties drastically (it is tunable). 4G) In an alternative embodiment, the mechanical properties were further tuned by crosslinking collagen tubes by either vitrification (a process that lets collagen dry at a controlled temperature and humidity that causes collagen fibers to crosslink) or treating with an external crosslinking agent-(3-[Tris(hydroxymethyl)phosphonio propionate] or THPP. 4H) THPP cross liking enhanced the Young's modulus and breaking stress of the molded collagen structures, although with compromised elongation (%) value. 4I) Radial burst pressure strength and volume expansion of the vitrified condensed tubes.

FIGS. 5A-5J depict tunable design and structures. Collagen articles with a plethora of design possibilities in both 5A) longitudinal and 5B) cross-sectional directions with various 5C) complex design features, such as tubes with corrugated surface, a tubular diversion, duckpin-like structure and mushroom-like to 5D) uniformly circular, pentagonal, multi-folded, trapezoid-like, octagonal-shaped and multi-channel molded tubes were created. 5E) Tubular scaffolds with varying dimensions, diameters ranging from ˜1 cm to 300-700 μm and length up to 7-8 cm, were developed (scale bar=250 μm). Material properties, such as 5F) kink-ability, 5G) stretchability of and 5H) porosity were imparted with in the molded collagen tubes. 5I) Furthermore, tubular scaffolds with an alternating multi-layered arrangement of condensed and porous layers (scale bar=2.0 mm). 5J) Three-D printed molds and corresponding tubular scaffolds with villi (image in bottom, flipped inside out for a better illustration as indicated by an arrow), and with negative impression of villi (image in top, cut to show the inner design and indicated by an arrow) (scale bar=1.0 cm). 5K) Alveolar sac-like scaffold (design, mold and final collagen scaffold) can be potentially developed & evaluated for their biological performance (scale bar=2.5 mm).

FIGS. 6A-6L depict in vitro stem cell culture & differentiation. 6A) MSCs seeded on condensed scaffolds without any external crosslinking agents, are viable and proliferating throughout the scaffold after 72 h of seeding. 6B) Hydrothermally crosslinked (vitrification) tubular scaffolds seeded with hADSCs are viable overtime as shown by the live-dead staining (electrospun PLGA as a control). 6C) Collagen (+PRP) scaffolds facilitate cell proliferation, while the number of cells decreased overtime in PLGA scaffolds, possibly due to degradation of PLGA scaffold and local increase in pH. 6D) SEM images showed a layer-by-layer arrangement of dense collagen fibers in hydrothermally cross-linked tubular scaffolds, and as expected cells grew mostly on top of the surface. To facilitate cell penetrations and increase the overall surface area reachable to cells, porous collagen tubular scaffolds were designed by adding porogens. 6E) SEM showed porous structures of the tubes across the cross-sectional plane. Cells proliferated on the peripheral surface and spread across the pores of the tubes and more profoundly in PRP-containing scaffolds. The developed porous tubes were mechanically strong and compliant: stress-strain curve in 6F) longitudinal, 6G) transverse planes, and 6H) radial burst pressure strengths vs. volume expansion. In addition, the fabrication process enables us to directly embed cells while condensing collagen. 6I) Embedded cells were viable even after 7 days of culture. Confocal images of embedded hMSCs showed a spatial arrangement of the cells across the center and along the longitudinal axis of the tube. 6J) H&E staining shows the elongated morphology of the cells at the outer periphery while more round cell morphology toward inner periphery. 6K) Embedded-hADSCs cultured in smooth muscle cell differentiation medium, and 6L) hUCs cultured in a mixed medium in the inner lumen of hSMCs seeded scaffolds showed upregulation of SMCs and urothelial genes, respectively.

FIGS. 7A-7J depict design and dimensions of the mold that was either 3D printed or assembled from carbon/polymer lead or polystyrene thin rods for creating designer scaffolds.

FIG. 8 shows a design of the porous mold for alveolar-sac like scaffolds. Design and dimensions of the mold that was 3D printed for creating alveolar-sac like scaffolds.

### DETAILED DESCRIPTION OF THE INVENTION

In the era of employing plethora of chemistry design and physical parameters, such as controlled shape, mechanical properties, and surface topography to augment tissue repair and regeneration process, the innovation in processing design for developing mechanically robust yet biologically functional collagen materials is long overdue. A novel methodology that can facilitate molding collagen into various shapes and structures with sufficient mechanical properties can change the horizon of reconstructing and regenerating biologically functional tissues that otherwise have been difficult to engineer, including hollow and tubular urological system. It is critically relevant particularly if the molded tissue scaffolds are designed to be continuous and seamless structures, overcoming the mechanically weak points in the scaffolds.

In accordance with an embodiment, the present invention provides a mold apparatus (10) for making a molded tissue scaffold comprising an inlet/outlet adaptor (11), wherein said inlet/outlet adaptor (11) comprises an inlet port (12) and an outlet port (13) which can allow fluids and gases to pass through the inlet (12) or outlet port (13) of the inlet/outlet adaptor (11), said inlet/outlet adaptor (11) further comprising an internal mold element (14) comprised of a sintered material which is semi-permeable or a porous material and said internal mold element defining a hollow interior space which connects to the outlet port (13) of the inlet/outlet adaptor (11) and communicates with the outlet port (13) of the inlet/outlet adaptor, said internal mold element is capable of allowing gas and fluid to pass through the exterior of the internal mold element into the hollow interior space of the internal mold element and exit out of the outlet port (13) of the inlet/outlet adaptor (11); the mold apparatus (10) further comprises a mold chamber (15) which is comprised of at least one wall (16) comprising a flexible material which defines the inside and outside of the mold chamber (15), and encloses the internal mold element (14), and which is fastened at one end, to the inlet/outlet adaptor (11); the inlet port (12) of the at least first adaptor communicates with the interior of the mold chamber (15) such that fluid and a liquid tissue composition can enter into the mold chamber (15) and be contained within said chamber; the liquid tissue composition can be added to the chamber via the inlet port (12) at sufficient pressure to expand the flexible wall (16) of the mold chamber (15) such that the wall of the mold chamber (15) will provide counter pressure to the liquid in the mold chamber (15) and press against the internal mold element.

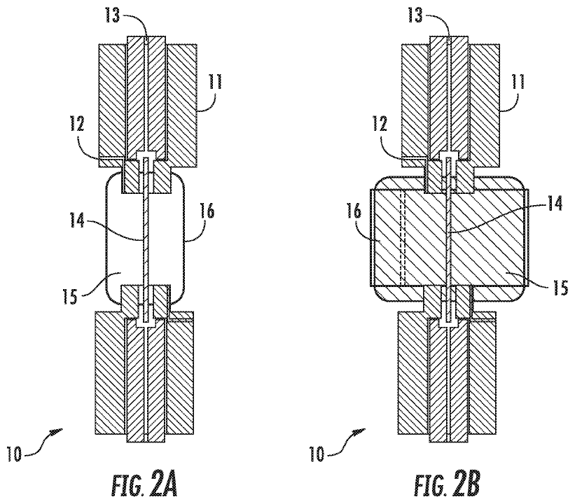

FIG. 2 depicts an embodiment of the mold apparatus used in the inventive methods. FIG. 2A depicts the mold apparatus with a tubular internal mold element as ready for filling with a soluble tissue scaffold solution. FIG. 2B depicts the apparatus filled with a soluble tissue scaffold solution and prepared to condense the solution into a solid matrix. The apparatus can comprise one or more adaptors which communicate with a mold chamber. In FIG. 2, an embodiment of the apparatus is depicted with an upper and lower adaptor which are identical. The mold apparatus comprises at least one adaptor which comprises at least one or more inlet ports and at least one or more outlet ports. The inlet ports, in some embodiments, can be adapted to connect to common laboratory fluid handling equipment, such as syringes (e.g., via luer lock) or with fittings used for pumps such as dialysis pumps having low pressure. It is envisioned that one can devise a set up with multiple holders and unique flexible material design, such as a multi mouth balloon shaped scaffold.

In accordance with an alternative embodiment, instead of a semi-permeable or porous internal mold element, one can use a solid impermeable internal mold element. In this embodiment, the molding process becomes more dependent on the force that the flexible wall of the mold chamber imparts to the tissue scaffold solution during the vitrification/fibrinogenesis process. In this embodiment, the water in the tissue scaffold solution can exit the mold chamber via the inlets and in some embodiments, such as in the embodiment where the apparatus comprises two adaptors, gravity can assist in removal of water from the scaffold solution via exiting out the inlet of the lower adaptor.

In some embodiments the internal mold element which is semi-permeable/porous, can be dissolvable or sacrificial or solid (as tubes are easy to remove). For hollow spherical shapes, such as for bladder, one can use porous sacrificial/dissolvable or tiny spherical mold elements like pebbles combined with adhesive polymers. Once the hollow bladder like structure is formed, then the adhesive polymer can be sacrificed/dissolved and tiny pebbles can be drained out from the hollow scaffold.

In some embodiments, the mold apparatus (10) can comprise at the other end opposite of the inlet/outlet adaptor (11), to either a plug or impermeable wall, or as shown in FIG. 2, a second adaptor or it can be non-existent (13).

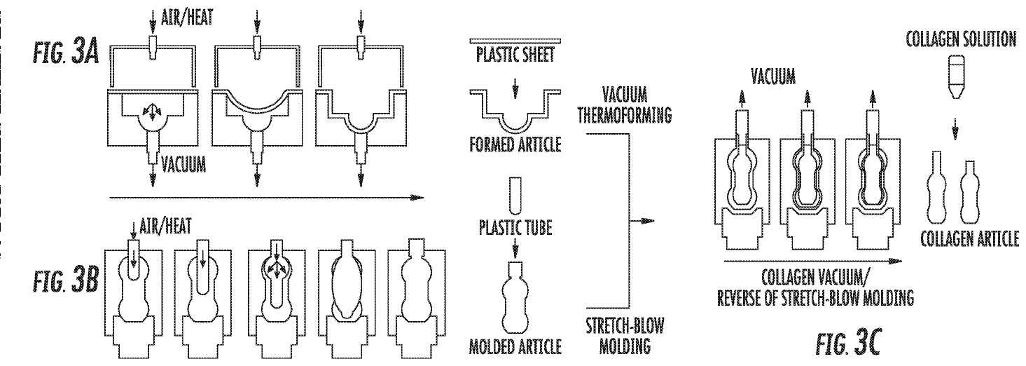

In some embodiments, for example, as in embodiments such as for use creating a hollow article, such as a bladder, the mold apparatus (10) can comprise a bulbar or concave internal mold element (14) (see, FIG. 3B) wherein the mold apparatus (10) comprises only a single inlet/outlet adaptor (11), and the flexible wall (16) of the mold chamber (15) has a spherical or balloon shape attached to the inlet/outlet adaptor (11).

In some embodiments, the inlet/outlet adaptor can be made from any rigid durable materials such as stainless steel, plastic, or glass. It will be understood that the inlet/outlet adaptor can be in other configurations besides those depicted herein, and can be modified to create tissue scaffolds of various shapes and sizes. Moreover, it will also be understood that the internal mold elements contemplated herein, can have any shape, as needed to replicate an existing organ shape and size.

The inlet and outlet of the inlet/outlet adaptor can be adapted to connect to other apparatus via readily available fittings for use in transfer of liquids and gases. For example, the outlet can be adapted for use in applying vacuum to the mold apparatus for condensing the tissue scaffold solution and allowing fibrogenesis and vitrification.

In some embodiments it is contemplated that the flexible wall (16) of the mold chamber (15) is translucent or permeable to wavelengths of light which can allow initiation of cross-linking of the tissue scaffold solution, such as UV or infrared wavelengths of light.

As used herein, the term “mold chamber” means a flexible, expandable container having an inlet/outlet, and an internal mold element, the container being capable of containing the protein solution in and/or around the internal mold element under pressure and gravity. The container and the internal mold element can have any shape and the container or mold chamber comprises at least one inlet/outlet for adding the protein solution. In some embodiments, the flexible wall (16) of the mold chamber (15) is comprised of any durable, flexible materials, such as natural or synthetic rubber or synthetic polymers such as, ethylene propylene diene monomer (EPDM) or a recently developed Panasonic resin stretchable film that is based on soft and rigid polymeric domains

(news.panasonic.com/global/press/data/2015/12/en151224-3/en151224-3.html).

In accordance with an embodiment, the present invention provides a method for making a molded tissue scaffold comprising the steps of: a) solubilizing a solution comprising one or more fibrous proteins suitable for use as a tissue scaffold; b) combining the solution of a) with at least a second solution which will promote fibrogenesis and vitrification of the protein solution of a); c) adding the combined solution of b) into the inlet of a mold capable of containing the solution of b) under pressure and gravity, and which comprises an internal mold element which is semi-permeable and communicates at one end to the outlet of the mold apparatus; d) condensing the solution of b) in the expandable mold chamber of the mold apparatus of c; until the scaffold has sufficient wall thickness and tensile strength; and e) removal of the molded tissue scaffold from the mold.

In some embodiments, the method can include the additional steps of further biological and mechanical processing, such as crosslinking.

In some embodiments, the inventive method further comprises condensing the tissue scaffold solution via application of vacuum to the outlet of the mold apparatus and/or pressure from the expandable mold chamber pressing the tissue scaffold solution against the internal mold element, and removing water from the solution of b) until the scaffold has sufficient tensile strength; and e) removal of the molded tissue scaffold from the mold apparatus.

In some embodiments, the tissue scaffold solution can be condensed via cross-linking via application of UV or infrared light to the mold chamber (15).

In some embodiments, cells, such as MSCs, hADSCs and other cells can be added to the tissue scaffold solution in the mold chamber prior to vitrification.

Thus, in accordance with some embodiments, the inventive apparatus and methods provide novel tissue scaffold molding technology processes which reconfigures modalities of commonly employed plastic “thermoforming and blow molding” techniques in a novel and unique way. In thermoforming (FIG. 3A), a heated plastic sheet is collapsed onto a pre-defined internal mold by applying vacuum to form the plastic into a desired shape, while in stretch-blow molding (FIG. 3B) a hollow article is created by forcing air from inside of a heated plastic tube that stretches and presses the blown tube against the internal mold of pre-defined shape.

In accordance with the inventive apparatus and methods, in order to develop hollow or concave and tubular tissue scaffold structures, the present inventors reconfigured the concepts of vacuum thermoforming and stretch-blow molding; although, in this process a solubilized fibrous protein solution, such as collagen, is condensed and pressed against an internal mold element by applying vacuum or negative pressure in contrast to molding a thermoplastic where the solid plastic tube or sheet is soften and stretched (FIG. 3C).

As used herein, the term “a solution comprising one or more fibrous proteins suitable for use as a tissue scaffold” means any biocompatible fibrous protein or proteins which can be formed using the inventive methods. In an embodiment, the solution comprising one or more fibrous proteins using the methods of the present invention can comprise collagen. The collagen can be selected from the group consisting of Type I, Type II, Type III and Type IV collagen and mixtures thereof. In an embodiment, the collagen used is Type I collagen. One of ordinary skill in the art would understand that the collagen used in the compositions and methods could include more than one type of collagen.

It will be understood that other proteins other than collagen can be used in the inventive apparatus and methods disclosed herein. Examples of such proteins include, for example, fibronectin, actin, cadherin, fibrin, heparin, laminin, myelin, troponin, tubulin and the like.

In accordance with other embodiments, the tissue scaffold solution comprising one or more fibrous proteins can be combined with other tissue scaffold components, such as, for example, biologically compatible polymers, such as hyaluronic acid, chondroitin sulfate, fibrinogen, albumin, elastin, synthetic peptides, synthetic polymers, such as poly(ethylene glycol), micro/nanoparticles, and decellularized tissue components.

A biologically compatible polymer refers to one that is a naturally occurring polymer or one that is not toxic to the host. The polymer can, e.g., contain at least an imide. The polymer may be a homopolymer where all monomers are the same or a hetereopolymer containing two or more kinds of monomers. The terms “biocompatible polymer,” “biocompatible cross-linked polymer matrix” and “biocompatibility” when used in relation to the instant polymers are art-recognized are considered equivalent to one another, including to biologically compatible polymer. For example, biocompatible polymers include polymers that are neither toxic to the host (e.g., an animal or human), nor degrade (if the polymer degrades at a rate that produces monomeric or oligomeric subunits or other byproducts at toxic concentrations in the host).

In some embodiments, a monomeric unit of a biologically compatible polymer may be functionalized through one or more thio, carboxylic acid or alcohol moieties located on a monomer of the biopolymer. For example, in the case of chondroitin sulfate, a carbonyl group can be derivatized with a imide group using, for example, carbodiimide chemistry. An alcohol group can be derivatized using, for example, the Mitsunobu reaction, Procter et al., Tetra. Lett. 47(29): 5151-5154, 2006.

In some embodiments, the tissue scaffold composition comprising at least one monomeric unit of a biologically compatible polymer, such as CS, hyaluronic acid, heparin sulfate, keratan sulfate and the like, functionalized by an imide. Those starting molecules are natural components of extracellular matrices. However, in general, any biologically compatible polymer can be used as the polymer, which polymer carries at least an imide. Other suitable polymers include those which are naturally occurring, such as a GAG, mucopolysaccharide, collagen or proteoglycan components, such as hyaluronic acid, heparin sulfate, glucosamines, dermatans, keratans, heparans, hyalurunan, aggrecan, and the like.

In some embodiments, this disclosure is directed to a tissue scaffold composition comprising at least one monomeric unit of a saccharide or other biocompatible monomer or polymer, wherein the monomers have reactive sites that will enable at least inclusion of an imide and other functional groups, such as chondroitin sulfate. Chondroitin sulfate is a natural component of cartilage and may be a useful scaffold material for regeneration. Chondroitin sulfate includes members of 10-60 kDa glycosaminoglycans. The repeat units, or monomeric units, of chondroitin sulfate consist of a disaccharide, 13(1→4)-linked D-glucuronyl 13(1→3)N-acetyl-D-galactosamine sulfate.

The tissue scaffold compositions of the present invention may comprise monomers, macromers, oligomers, polymers, or a mixture thereof. The polymer compositions can consist solely of covalently crosslinkable polymers, or ionically crosslinkable polymers, or polymers crosslinkable by redox chemistry, or polymers crosslinked by hydrogen bonding, or any combination thereof. The reagents should be substantially hydrophilic and biocompatible.

Suitable hydrophilic polymers which can be incorporated into the molded tissue scaffold include synthetic polymers such as poly(ethylene glycol), poly(ethylene oxide), partially or fully hydrolyzed poly(vinyl alcohol), poly(vinylpyrrolidone), poly(ethyloxazoline), poly(ethylene oxide)-co-poly(propylene oxide) block copolymers (poloxamers and meroxapols), poloxamines, carboxymethyl cellulose, and hydroxyalkylated celluloses such as hydroxyethyl cellulose and methylhydroxypropyl cellulose, and natural polymers such as polypeptides, polysaccharides or carbohydrates such as Ficoll™, polysucrose, hyaluronic acid, dextran, heparan sulfate, chondroitin sulfate, heparin, or alginate, and proteins such as gelatin, collagen, albumin, or ovalbumin, carboxy methyl starch, or copolymers or blends thereof. As used herein, “celluloses” includes cellulose and derivatives of the types described above; “dextran” includes dextran and similar derivatives thereof.

The fibrous proteins are solubilized using known means of acidification. Generally, the protein solution is prepared using lyophilized protein, such as collagen, in HCl at a concentration of 1 to 10 mg/ml, preferably about 5 mg/ml. When the proteins are dissolved, the solution is then neutralized using a second solution which promotes fibrogenesis.

In an alternate embodiment, that tissue scaffold solution can be made by dissolution of a protein matrix in a fluorocarbon solvent, such as 1,1,1,3,3,3-hexafluoro-2-propanol.

As used herein the term “second solution which promotes fibrogenesis” means a buffering agent in a biologically compatible buffer. For example, the protein solution is then added to a second solution comprising cell culture medium which contains a buffering agent that is biocompatible, such as HEPES and kept at 4° C. The solution is mixed and then added to the apparatus having an internal mold chamber (15). It is expected that there can be a variety of neutralizing collagen solution that can induce fibrogenesis of collagen, e.g. a sodium hydroxide solution can be used to neutralize collagen dissolved in acetic acid or hydrochloric acid.

As used herein, the term “vitrification” or “vitrigel” means that the composition is composed of an aqueous solution of a mixture of one or more tissue scaffold proteins, such as collagens and allowed to form a hydrogel. In some embodiments, the gelation of the composition is performed at a temperature of 37° C. After the hydrogel is formed, the hydrogel is vitrified by dehydration, such as, for example, heating the hydrogel at a specific temperature and humidity, for a specific length of time to allow vitrification to occur. In some embodiments, the vitrification is performed at a temperature of 35 to 45° C. and a humidity of between about 30% and 50% relative humidity. In an embodiment, the vitrification is performed at a temperature of 40° C. and a relative humidity of 40%. The time needed for vitrification of the compositions can vary from a few days to a few weeks. In an embodiment, the time for vitrification of the compositions is from a few minutes to, 1 h to 1 day to 2 weeks.

“Gel” refers to a state of matter between liquid and solid, and is generally defined as a cross-linked polymer network swollen in a liquid medium. Typically, a gel is a two-phase colloidal dispersion containing both solid and liquid, wherein the amount of solid is greater than that in the two-phase colloidal dispersion referred to as a “sol.” As such, a “gel” has some of the properties of a liquid (i.e., the shape is resilient and deformable) and some of the properties of a solid (i.e., the shape is discrete enough to maintain three dimensions on a two-dimensional surface).

By “hydrogel” is meant a water-swellable polymeric matrix that can absorb water to form elastic gels, wherein “matrices” are three-dimensional networks of macromolecules held together by covalent or noncovalent crosslinks. On placement in an aqueous environment, dry hydrogels swell by the acquisition of liquid therein to the extent allowed by the degree of cross-linking.

In some embodiments, the second solution can comprise cross-linking agents. Cross-linked herein refers to a composition containing intermolecular cross-links and optionally intramolecular cross-links, arising from, generally, the formation of covalent bonds. Covalent bonding between two cross-linkable components may be direct, in which case an atom in one component is directly bound to an atom in the other component, or it may be indirect, through a linking group. A cross-linked gel or polymer matrix may, in addition to covalent, also include intermolecular and/or intramolecular noncovalent bonds such as hydrogen bonds and electrostatic (ionic) bonds.

Cross-linking can be initiated by many physical or chemical mechanisms. Photopolymerization is a method of covalently crosslink polymer chains, whereby a photoinitiator and polymer solution (termed “pre-gel” solution) are exposed to a light source specific to the photoinitiator. On activation, the photoinitiator reacts with specific functional groups in the polymer chains, crosslinking them to form the hydrogel. The reaction is rapid (3-5 minutes) and proceeds at room and body temperature. Photoinduced gelation enables spatial and temporal control of scaffold formation, permitting shape manipulation after injection and during gelation in vivo. Cells and bioactive factors can be easily incorporated into the hydrogel scaffold by simply mixing with the polymer solution prior to photogelation.

It will be understood that the molded tissue scaffold compositions of the present invention can be molded or formed into any particular shape, including hollow structures, suitable for use as a replacement tissue or tissue filler. The instant invention provides for ex vivo polymerization techniques to form scaffolds and so on that can be molded to take the desired shape of a tissue defect, promote tissue development by stimulating native cell repair, and can be implanted by surgical methods.

In accordance with another embodiment, the present invention provides a composition comprising a molded tissue scaffold having a first component and at least one second component, wherein the first component comprises a tissue scaffold protein or proteins, such as collagen, and wherein the second component comprises at least one biologically active agent.

An “active agent” and a “biologically active agent” are used interchangeably herein to refer to a chemical or biological compound that induces a desired pharmacological and/or physiological effect, wherein the effect may be prophylactic or therapeutic. The terms also encompass pharmaceutically acceptable, pharmacologically active derivatives of those active agents specifically mentioned herein, including, but not limited to, salts, esters, amides, prodrugs, active metabolites, analogs and the like. When the terms “active agent,” “pharmacologically active agent” and “drug” are used, then, it is to be understood that the invention includes the active agent per se as well as pharmaceutically acceptable, pharmacologically active salts, esters, amides, prodrugs, metabolites, analogs etc.

Incorporated,” “encapsulated,” and “entrapped” are art-recognized when used in reference to a therapeutic agent, dye, or other material and the molded tissue scaffold of the present invention. In certain embodiments, these terms include incorporating, formulating or otherwise including such agent into a composition that allows for sustained release of such agent in the desired application. The terms may contemplate any manner by which a therapeutic agent or other material is incorporated into a matrix, including, for example, distributed throughout the matrix, appended to the surface of the matrix (by intercalation or other binding interactions), encapsulated inside the matrix, etc. The term “co-incorporation” or “co-encapsulation” refers to the incorporation of a therapeutic agent or other material and at least one other therapeutic agent or other material in the molded tissue scaffold composition.

The biologically active agent may vary widely with the intended purpose for the composition. The term active is art-recognized and refers to any moiety that is a biologically, physiologically, or pharmacologically active substance that acts locally or systemically in a subject. Examples of biologically active agents, that may be referred to as “drugs”, are described in well-known literature references such as the Merck Index, the Physicians' Desk Reference, and The Pharmacological Basis of Therapeutics, and they include, without limitation, medicaments; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment. Various forms of a biologically active agent may be used which are capable of being released by the molded tissue scaffold composition, for example, into adjacent tissues or fluids upon implantation into a subject.

Various forms of the biologically active agents may be used. These include, without limitation, such forms as uncharged molecules, molecular complexes, salts, ethers, esters, amides, prodrug forms and the like, which are biologically activated when implanted, injected or otherwise placed into a subject.

For example, a therapeutic agent, biologically active agent, or other chemical moiety attached as a side chain to the polymer backbone may be released by biodegradation. In certain embodiments, one or the other or both general types of biodegradation may occur during use of a polymer. As used herein, the term “biodegradation” encompasses both general types of biodegradation.

The degradation rate of a biodegradable polymer often depends in part on a variety of factors, including the chemical identity of the linkage responsible for any degradation, the molecular weight, crystallinity, biostability, and degree of cross-linking of such polymer, the physical characteristics of the implant, shape and size, and the mode and location of administration. For example, the greater the molecular weight, the higher the degree of crystallinity, and/or the greater the biostability, the biodegradation of any biodegradable polymer is usually slower. The term “biodegradable” is intended to cover materials and processes also termed “bioerodible.”

In some embodiments, a biologically active agent may be used in cross-linked polymer matrix of this invention, to, for example, promote cartilage formation. In other embodiments, a biologically active agent may be used in cross-linked polymer matrix of this invention, to treat, ameliorate, inhibit, or prevent a disease or symptom, in conjunction with, for example, promoting cartilage formation.

Further examples of biologically active agents include, without limitation, enzymes, receptor antagonists or agonists, hormones, growth factors, autogenous bone marrow, antibiotics, antimicrobial agents, and antibodies. The term “biologically active agent” is also intended to encompass various cell types and genes that can be incorporated into the compositions of the invention.

In certain embodiments, the subject compositions comprise about 1% to about 75% or more by weight of the total composition, alternatively about 2.5%, 5%, 10%, 20%, 30%, 40%, 50%, 60% or 70%, of a biologically active agent.

Non-limiting examples of biologically active agents include following: adrenergic blocking agents, anabolic agents, androgenic steroids, antacids, anti-asthmatic agents, anti-allergenic materials, anti-cholesterolemic and anti-lipid agents, anti-cholinergics and sympathomimetics, anti-coagulants, anti-convulsants, anti-diarrheal, anti-emetics, anti-hypertensive agents, anti-infective agents, anti-inflammatory agents such as steroids, non-steroidal anti-inflammatory agents, anti-malarials, anti-manic agents, anti-nauseants, anti-neoplastic agents, anti-obesity agents, anti-parkinsonian agents, anti-pyretic and analgesic agents, anti-spasmodic agents, anti-thrombotic agents, anti-uricemic agents, anti-anginal agents, antihistamines, anti-tussives, appetite suppressants, benzophenanthridine alkaloids, biologicals, cardioactive agents, cerebral dilators, coronary dilators, decongestants, diuretics, diagnostic agents, erythropoietic agents, estrogens, expectorants, gastrointestinal sedatives, agents, hyperglycemic agents, hypnotics, hypoglycemic agents, ion exchange resins, laxatives, mineral supplements, mitotics, mucolytic agents, growth factors, neuromuscular drugs, nutritional substances, peripheral vasodilators, progestational agents, prostaglandins, psychic energizers, psychotropics, sedatives, stimulants, thyroid and anti-thyroid agents, tranquilizers, uterine relaxants, vitamins, antigenic materials, and prodrugs.

In accordance with an alternative embodiment, porous molded tubular scaffolds, made with collagen, were designed by adding porogens as the second active agent to the tissue scaffold solution. Examples of porogens include camphor microparticles (for example, filtered 250 um size particles; concentration 1 gm/25 mg of collagen). It is expected that other porogen can be used to create porosity, such as menthol or effervescent such as, coated ammonium carbonate powder or particles that releases CO₂ above room temperature (˜40 degree centigrade).

In one embodiment, the repair of damaged tissue, such as a bladder or ureter may be carried out within the context of any standard surgical process allowing access to and repair of the tissue, including open surgery and laparoscopic techniques. Once the damaged tissue is accessed, a molded tissue scaffold composition of the invention is placed in contact with the damaged tissue along with any surgically acceptable patch or implant, if needed.

The term, “carrier,” refers to a diluent, adjuvant, excipient or vehicle with which the therapeutic is supplied with the vitrigel composition of the present invention. Such physiological carriers can be sterile liquids, such as water and oils, including those of petroleum, animal, vegetable or synthetic origin, such as peanut oil, soybean oil, mineral oil, sesame oil and the like. Water is a suitable carrier when the pharmaceutical composition is administered intravenously. Saline solutions and aqueous dextrose and glycerol solutions also can be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents.

Buffers, acids and bases may be incorporated in the compositions to adjust pH. Agents to increase the diffusion distance of agents released from the composition may also be included.

Buffering agents help to maintain the pH in the range which approximates physiological conditions. Buffers are preferably present at a concentration ranging from about 2 mM to about 50 mM. Suitable buffering agents for use with the instant invention include both organic and inorganic acids, and salts thereof, such as citrate buffers (e.g., monosodium citrate-disodium citrate mixture, citric acid-trisodium citrate mixture, citric acid-monosodium citrate mixture etc.), succinate buffers (e.g., succinic acid monosodium succinate mixture, succinic acid-sodium hydroxide mixture, succinic acid-disodium succinate mixture etc.), tartrate buffers (e.g., tartaric acid-sodium tartrate mixture, tartaric acid-potassium tartrate mixture, tartaric acid-sodium hydroxide mixture etc.), fumarate buffers (e.g., fumaric acid-monosodium fumarate mixture, fumaric acid-disodium fumarate mixture, monosodium fumarate-disodium fumarate mixture etc.), gluconate buffers (e.g., gluconic acid-sodium glyconate mixture, gluconic acid-sodium hydroxide mixture, gluconic acid-potassium gluconate mixture etc.), oxalate buffers (e.g., oxalic acid-sodium oxalate mixture, oxalic acid-sodium hydroxide mixture, oxalic acid-potassium oxalate mixture etc.), lactate buffers (e.g., lactic acid-sodium lactate mixture, lactic acid-sodium hydroxide mixture, lactic acid-potassium lactate mixture etc.) and acetate buffers (e.g., acetic acid-sodium acetate mixture, acetic acid-sodium hydroxide mixture etc.). Phosphate buffers, carbonate buffers, histidine buffers, trimethylamine salts, such as Tris, HEPES and other such known buffers can be used.

Examples of diluents include a phosphate buffered saline, buffer for buffering against gastric acid in the bladder, such as citrate buffer (pH 7.4) containing sucrose, bicarbonate buffer (pH 7.4) alone, or bicarbonate buffer (pH 7.4) containing ascorbic acid, lactose, or aspartame. Examples of carriers include proteins, e.g., as found in skim milk, sugars, e.g., sucrose, or poly(vinyl pyrrolidone). Typically these carriers would be used at a concentration of about 0.1-90% (w/v) but preferably at a range of 1-10%.

### General Description of Shapes of Molded Tissue Scaffolds Which can be Produced and Methods of Production

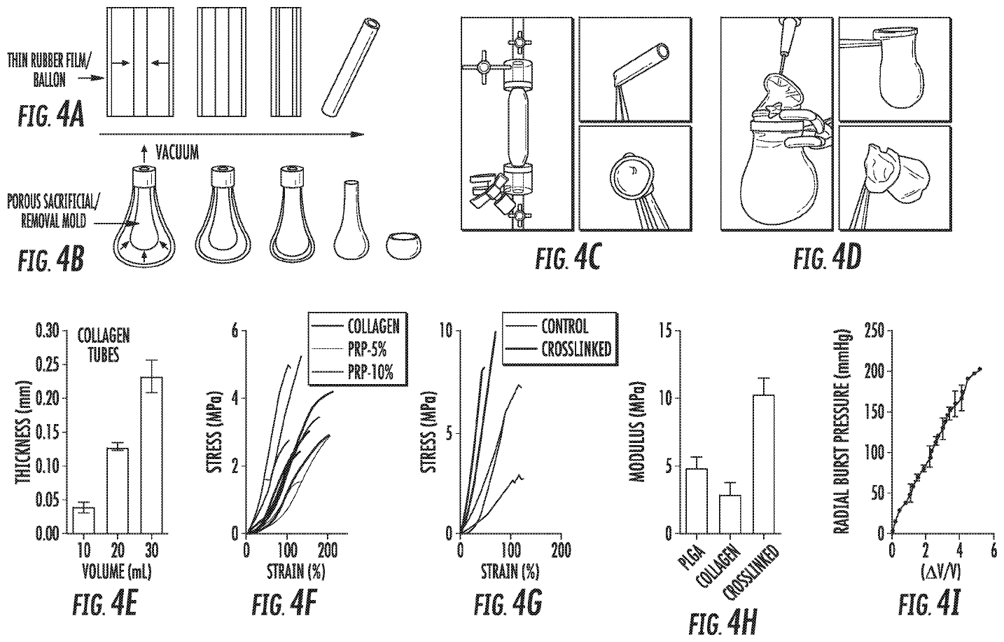

To develop various concave or hollow and tubular molded tissue scaffold structures, the protein substrate used in the production of the molded tissue scaffold, for example, collagen, is first solubilized, and then placed in the mold apparatus. The soluble tissue scaffold solution is then condensed and pressed against the internal mold by applying vacuum or negative pressure in contrast to molding a thermoplastic where the solid plastic tube or sheet is soften and stretched (FIG. 2C). Specifically, the present inventors have developed collagen scaffolds by injecting a freshly mixed ice-cold acid-solubilized collagen and its neutralizing aqueous solution into a thin rubber balloon (FIGS. 4A & B). The collagen-filled balloon itself can have a pre-defined shape or it can simply press the chamber with its unique design features for printing them on the molded articles. Critical to this process are the support of a thin flexible material, e.g. rubber or other stretchable or flexible balloon that holds the collagen fibrogenesis solution under fluid-pressure and the gravity, and the shape-providing porous sacrificial or detachable internal mold, which assists in expediting water extraction, further condensing the collagen solution on fibrogenesis under controlled vacuum or negative pressure.

In accordance with some embodiments, the shapes of the molded tissue scaffolds produced can be ultimately any 3-dimensional shape. Examples of shapes include, but are not limited to, tubular with uniform diameter, tubular with changing diameter, tubular with villi like protrusions, multi-layer (layer by layer), porous, dense, hollow-like spherical, diversion conduit, and ridged structures. These structures are dependent on the design of sacrificial or dissolvable internal mold element. The outer shape of the scaffold can be impressed by the flexible wall of the mold chamber in which the tissue scaffold takes shape. Collagen, for example, once it undergoes fibrogenesis, keeps the shape quite well; however, it is possible to custom make tissue scaffolds with different shapes that will hold the shape of the flexible wall of the mold chamber.

### Modulating Mechanical and Biochemical Properties of the Molded Tissue Scaffolds

Tissue scaffold molding using the embodiments of the apparatus and methods of the present invention results can create tubular as well as partial or full bladder-like bulbular and hollow/concave structures that can be useful for neo urinary diversion conduit and bladder applications (FIGS. 4C & D) with user controlled mechanical properties and biochemical functionalities. The scaffold solution can be mixed with other biological polymers, decellularized ECM or materials that can change the biochemical properties as well as mechanical properties. This can be either physical entrapment or covalent chemical reaction. Tubes with various wall thicknesses corresponding to the initial volume load (FIG. 4E) can be crosslinked to modulate tensile modulus and strength, and added with other biological agents, polymers and cells to impart unique biological and compositional features along with the specific structural scaffold designs. For example, we created collagen tubular conduits doped with PRP that has several growth factors to promote vascularization, stem cells migration and recruitment and stimulate remodeling and healing the process; and molecules, such as hyaluronic acids (HA), without much compromising its overall mechanical properties while enhancing its biological modalities. Collagen tubes with 5-10% PRP maintained the mechanical properties (FIG. 4F) that can be further tuned by crosslinking collagen either by a simple thermal dehydration method-vitrification or by treating with external crosslinking agents, such as THPP (Biomacromolecules 13, 3912-3916 (2012)) (FIG. 4G). THPP cross-linking enhanced the Young's modulus and breaking stress although with a compromised % elongation value (<50-60%) (FIG. 4G). We further compared the mechanical performance of the molded tubes (FIGS. 4H&I), in terms of tensile modulus and radial burst pressure strength values to poly(lactic-co-glycolic acid) (PLGA) electrospun tubes (Tengion™) that degrade overtime in PBS losing its strength and modulus by 10 fold within 14 days (data not shown). The molded tubes surpassed the radial burst strength of the physiological ureter in human adults with a radial burst strength of ˜150-200 mmHg and a volume expansion of 2.3 folds per 100 mmHg pressure increase without any leakage or rupture (FIG. 4I). By appropriate wall thickness, crosslinking and biological factors, the tubes can further be modulated for its user-controlled biomechanical properties. The present inventors further demonstrate the versatility of the process by designing and developing a wide spectrum of shapes and structures with tunable physical biophysical properties.

### Designer Molded Tissue Scaffolds with Unprecedented Flexibilities

In accordance with one or more embodiments, the present invention provides molded tissue scaffolds comprising one or more fibrous proteins having the 3-dimensional shape of an organ of the body. In some embodiments, the fibrous proteins used the scaffolds are various known types of collagen, e.g. type I, II, etc. Other examples include, but are not limited to, elastin, keratin, muscle proteins and others.

In accordance with some embodiments, the fibrous proteins used in the scaffolds of the present invention are cross-linked.

In accordance with some embodiments, the molded tissue scaffolds of the present invention can optionally comprise biopolymers, cells, and extracellular matrix components (ECM). The ECM is composed of two main classes of macromolecules: proteoglycans (PGs) and fibrous proteins. The main fibrous ECM proteins are collagens, elastins, fibronectins and laminins. PGs fill the majority of the extracellular interstitial space within the tissue in the form of a hydrated gel. PGs have a wide variety of functions that reflect their unique buffering, hydration, binding and force-resistance properties. For example, in the kidney glomerular BM, perlecan has a role in glomerular filtration. By contrast, in ductal epithelial tissues, decorin, biglycan and lumican associate with collagen fibers to generate a molecular structure within the ECM that is essential for mechanical buffering and hydration and that, by binding GFs, provides an easy, enzymatically accessible repository for these factors.

In accordance with some embodiments, the molded tissue scaffolds of the present invention can optionally comprise at least one active or biologically active agent. In some embodiments, the at least one active agent is a drug, or growth factor, polymers, biopolymers, decellularized tissue particles, and florescent markers.

In accordance with some embodiments, the molded tissue scaffolds of the present invention can optionally comprise at least one or more mammalian cells. In some embodiments, the at least one or more mammalian cells are stem cells.

In accordance with one or more embodiments, the present invention provides molded tissue scaffolds wherein the molded tissue scaffold is in a shape selected from the group consisting of: a ureter, bladder, urethra, small intestine, and a blood vessel, although any 3-dimensional tubular or spheroid construct can be made using the apparatus and methods disclosed herein.

In accordance with some embodiments, the molded tissue scaffolds of the present invention can be used to surgically replace of an organ or tissue in a body of a subject in need thereof. In some embodiments,

[symbol]

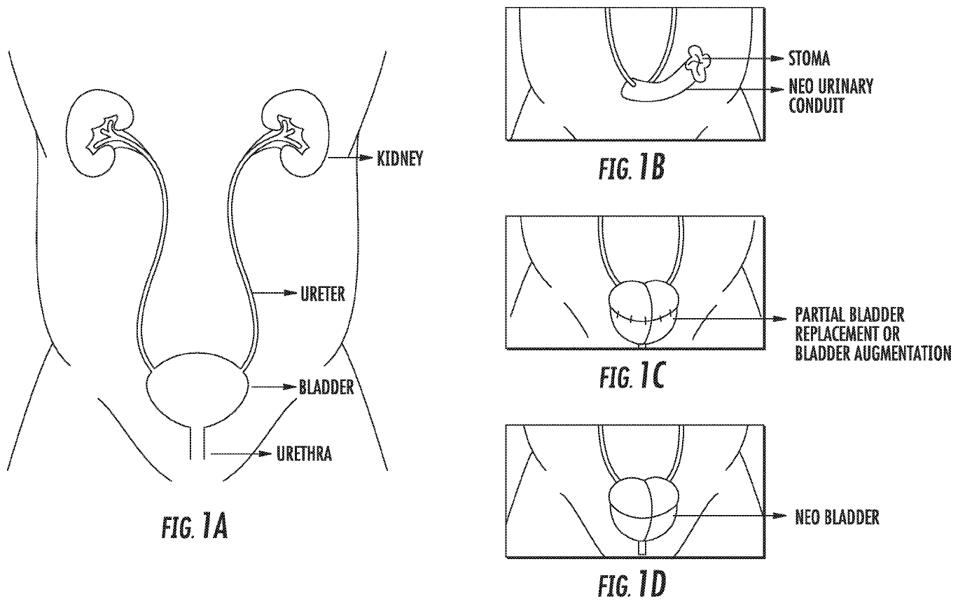

In accordance with some embodiments, the molded tissue scaffolds of the present invention can be used to surgically replace an organ which is diseased or non-functional, or which has a deformity or malformation due to a birth defect or genetic mutation. In other embodiments, the molded tissue scaffolds of the present invention can be used for ostomy or urethral replacement due to injury or disease. See, for example, FIG. 1B.

In accordance with some embodiments, the molded tissue scaffolds of the present invention can be used in the augmentation or supplementation of an organ in a body of a subject in need thereof. For example, a tubal structure could be used to extend a ureter which due to malformation, does not have proper orientation or implantation into the bladder of a subject. Other uses envisioned include, but are not limited to, new blood vessels in the heart or in a part of the body where the blood vessels were damaged due to injury or trauma.

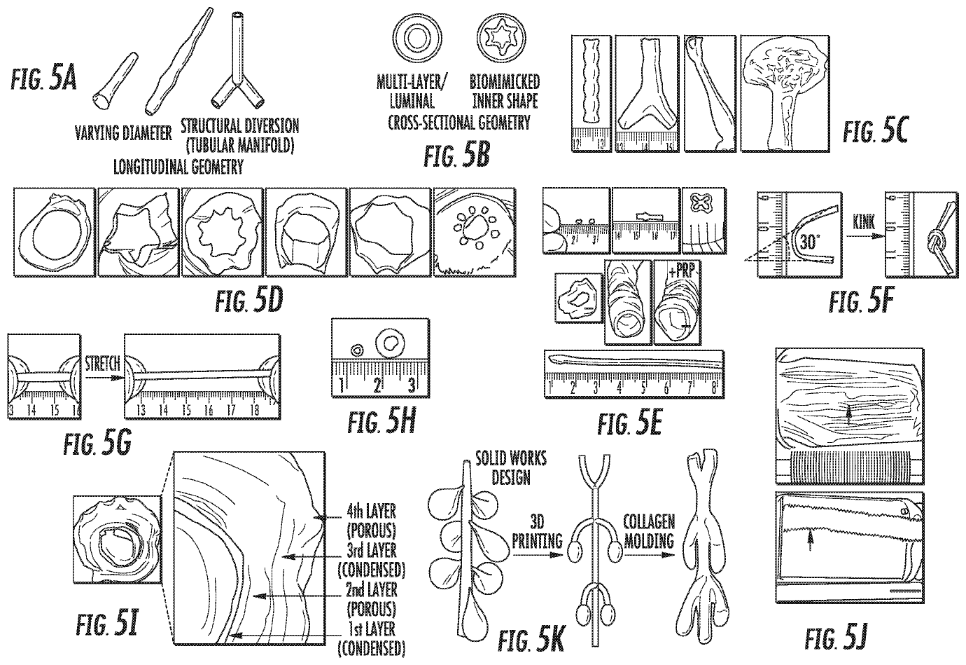

Urological tissues appear structurally uncomplicated; however, they are mechanically dynamic and biologically complex. Their structural design with a multi-folded inner lumen with enhanced surface area and a multi-layer muscle cell arrangement for contraction and expansion are critical for urine to flow without rupturing the lumen surface at a higher fluid shear stress. It gets even more complicated in the case of small intestine as its lumen is filled with micro protrusion known as villi, which are essential for nutrients absorption. For TE hollow or tubular neo-organs to be successful in pre-clinical and clinical settings, it is absolutely necessary to provide scaffolds with the desired shape and design with adequate mechanical strength required in reconstructed neo-tissues or neo-organs, which have not been possible to achieve using standard biomanufacturing techniques. Since the biofabrication process of the present invention utilizes attributes of vacuum thermoforming and stretch-blow molding plastic processing technologies, it can provide in countless shapes and design features. This enables the ability to create scaffolds with a plethora of design possibilities in both longitudinal and radial or cross-sectional planes. Therefore, using the inventive apparatus and methods, the inventors created tubular scaffolds that capture some of these design aspects of native ureters and intestinal features. The versatility of tissue scaffold molding further enabled us to create scaffolds with a plethora of design possibilities in both longitudinal and cross-sectional planes, including structural diversion or tubular manifold (FIGS. 5A,B).

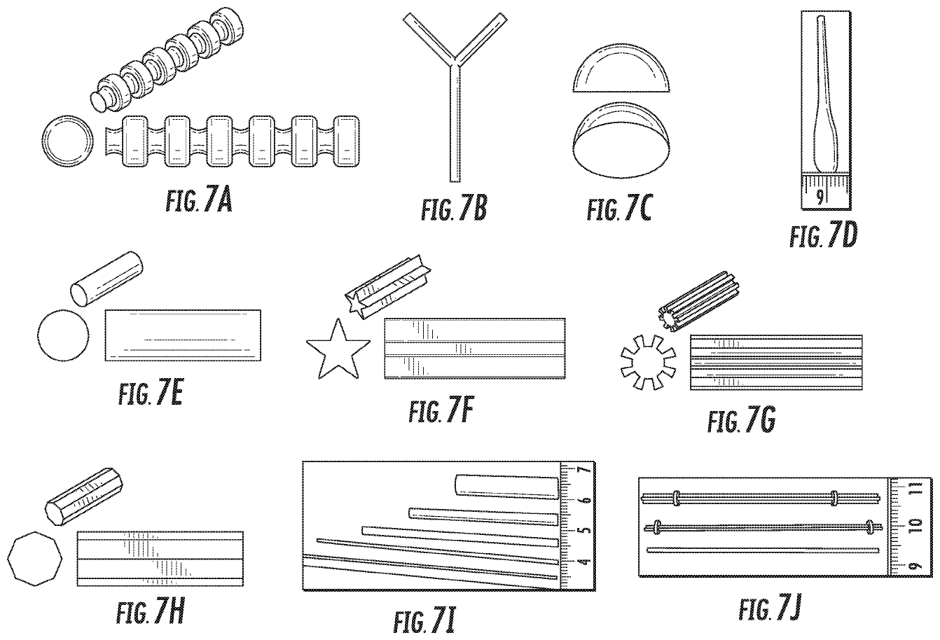

In accordance with alternative embodiments, it is also possible to create small diameter guiding channels to facilitate neural growth and blood supplies within the wall of the molded tubular tissue scaffolds. The present invention can provide, for example, tubular scaffolds with corrugated or vacuum-hose shape designs to enhance tube flexibility in the longitudinal direction (FIG. 5C, shown as a dry tube to be able to see corrugated texture), structural diversions or tubular manifolds to be able conjoin two tubular tissues and a duckpin shape with changing diameters in longitudinal axis (FIG. 5C), with diameter size varying from 100-500 μm to 1 cm and a full length of 7-8 cm. Tubes with micro-size diameters can be specifically developed for rodent urinary studies. FIGS. 7A-7J show exemplary molds that can be used with the inventive processes disclosed herein.

The present inventive methods allow creation of collagen tubes with changing shapes in the radial or cross-sectional direction (FIG. 5D, from circular to pentagonal star to octagonal star with folded sides to trapezoid to octagonal star to small diameter guiding channels in the tubular wall). It is noteworthy that the inventive methods provide the ability to create multi-folded cross-sectional tubular designs similar to the lumen of the ureter, and many possibilities can be investigated for their abilities to store and transfer urine with appropriate expansion ability in radial directions and without bursting under pressure. A tubular scaffold that is relatively stiff due to its inherent material composition can be made flexible enough to expand and contract solely using engineering design. Star-shaped tubes can expand and contract radially relatively easily compared to a round circular tubular structure without changing the material composition. In contrast, a corrugated tubular structure similar to a vacuum plastic hose can bend easily compared to other structures.

In accordance with some alternative embodiments, the present invention provides tubes with guiding channels of miniature diameters can potentially be used for facilitating neural growth and blood supplies (FIG. 5D). In another embodiment, the present invention provides designer micro tubes (also with embedded PRP), including multiple interconnected channels, with a full length up to 8.0 cm and diameter size up to 300 μm (as large as 1.0 cm diameter) that can be applied as either rodent urinary conduits or miniature blood vessels (FIG. 5E).

Another challenge with the existing PGA/PLGA conduits is the collapsed lumen with possible kinks on bending the engineered conduit during movement, which can occur when implanted through the abdominal wall/rectus muscle in humans to serve as a neo-urinary conduit. The miniature diameter tubes created using the inventive processes disclosed herein resisted kinking or bending up to ˜150° (180°-30°) of turn (FIG. 5F). In alternative embodiments, the kink angle can further be tuned by introducing corrugated surface or wrinkles similar to a rigid plastic-based vacuum hose that is extremely flexible due to its corrugated design (as shown in FIG. 5C) or by altering materials composition. Since material properties, such as stretchability and porosity are critical to conduits' biomechanical functions, i.e. volume compliance (volume expansion under urodynamic pressure), and cellular functions in terms of migration and proliferation, the inventive process disclosed herein creates highly stretchable tubular scaffolds by partially denaturing molded collagen tubes (FIG. 5G). Similarly, the inventive processes disclosed herein created porous scaffolds by adding a porogen (e.g. well-grinded camphor, <250 μm) to collagen solution before molding, which was sublimed off after molding the tube, leaving behind a porous scaffold (FIG. 5H). The inventive processes allowed the development of a multi-layered tubular scaffold with alternating porous and dense collagen layers (FIG. 5I). One of many practical implications of this design is in creating a tissue engineered collagen scaffold with a relatively compressible lumen yet stronger outer peripheral layer closely mimicking the multi-lamellar and multi-folded design feature of the physiological ureter.

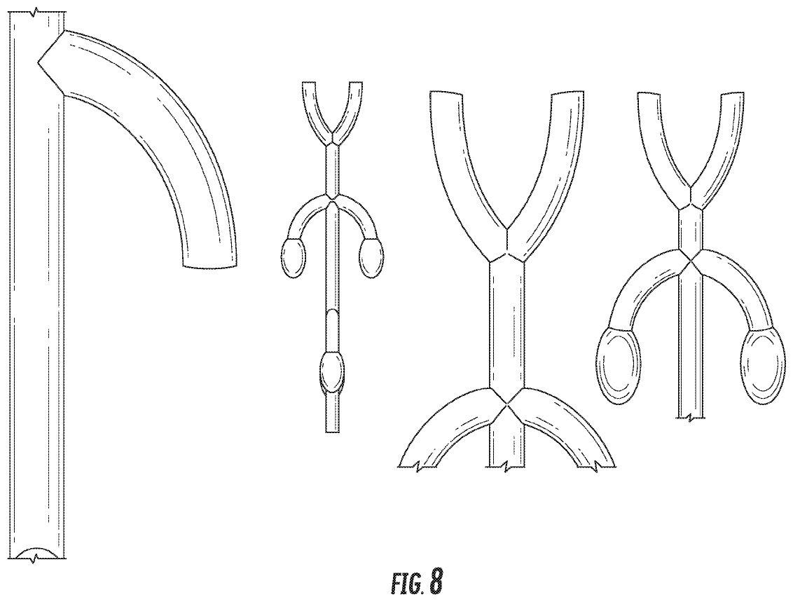

In accordance with some other embodiments, the present inventive methods provide collagen tubular-scaffolds with miniature design features similar to villi in the lumen of small intestine and the reverse or negative impression of villi as small troughs (FIG. 5J). In addition to genitourinary tissue engineering, gastrointestinal tissue engineering can also reap the benefits of this innovative process by employing thus developed single unit tubular scaffolds with well-defined structural design and mechanical properties. It is particularly interesting that scaffold's successful downstream application can eventually avoid a surgical excision of small segments of terminal ileum for recreating urinary segments. Furthermore, the application of the present inventive biomanufacturing process to create collagen scaffolds is not limited to only creating relatively simpler tubular designs, but a complex hollow design resembling alveolar sacs is also achievable (FIG. 5K, and FIG. 8 for examples of dimensions).

In accordance with an embodiment, the present invention provides methods for making molded tissue scaffolds is in the shape of an organ of the body. In some embodiments, the organ shapes can include, for example, a ureter, bladder, urethra, small intestine, and a blood vessel. Any shapes that can be made through the internal mold element and flexible wall of the mold chamber can be produced using the compositions and methods disclosed herein.

### Use of the Molded Tissue Scaffolds for In Vitro Stem Cell Culture & Differentiation

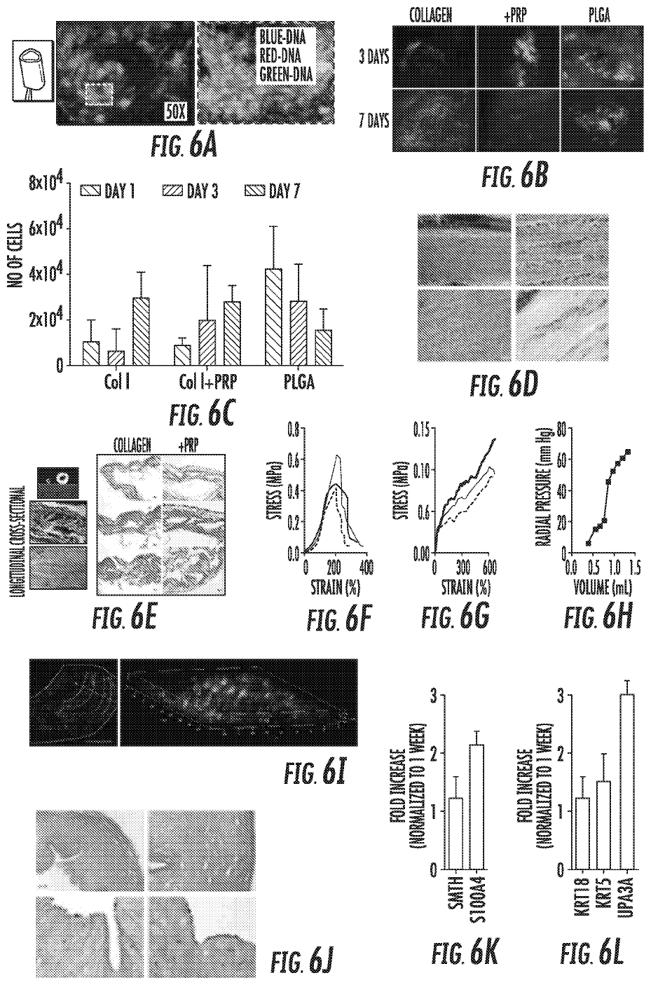

Mesenchymal stem cells (MSCs) seeded on molded tissue tubular scaffolds made with collagen using the inventive apparatus and methods, without external crosslinking agents or processes, were viable and proliferated throughout the scaffold after 72 h of seeding (FIG. 6A). Similarly, crosslinked molded tubular tissue scaffolds via vitrification, supported human adipose-derived stem cells (hADSCs) growth overtime as shown by the live-dead staining (FIG. 6B) (PLGA as a control).

In contrast to the molded tissue scaffolds made with collagen, the number of cells (quantified by an Alamar blue staining, FIG. 6C) decreased overtime on PLGA scaffolds, possibly due to degradation of PLGA scaffold and local increase in pH. SEM images showed layer-by-layer arrangement of dense collagen fibers in hydrothermally cross-linked molded tubular scaffolds of the present invention, where cells grew mostly on top of the surface (FIG. 6D).

To further facilitate cell penetrations and increase the overall surface area reachable to cells, in accordance with an alternative embodiment, porous molded tubular scaffolds, made with collagen, were designed by adding porogens (camphor, FIG. 6E). SEM showed porous structures of the tubes across the cross-sectional plane. Cells proliferated on the peripheral surface and spread across the pores of the molded tissue scaffold tubes although more profoundly in PRP-containing scaffolds possibly due to the less collagen density and biological growth factors (FIG. 6E).

In accordance with some embodiments, cells can be pre-mixed with the tissue scaffold solution prior to vitrification or fibrogenesis. Many different types of cells can be used, for example, hMSCs and chondrocytes have been successfully used in the present invention. Generally, one can mix the desired cell types with neutralized collagen solution and inject in the mold-chamber/set up and apply partial vacuum. While there is some cell death during the process, it is possible that one can optimize the parameters (like how much vacuum to apply for how long), and cells could be added with proteins like more FBS or hyaluronic acid that can help in cell survival of the solidification process.

In accordance with yet another embodiment, the present inventors successfully embedded cells while condensing the collagen tissue scaffold solution and created cell impregnated tubes under a partial vacuum that is viable even after 7 days of culture (FIG. 6I). Confocal images of embedded hMSCs showed spatial arrangement of the cells across the center of the tube. H&E staining shows the elongated morphology of the cells at the outer periphery, while more round cell morphology toward inner periphery (FIG. 6J). Seeded urothelial cells after 7 days of embedded hSMCs culture, proliferated at the peripheral-surface of the tube that can be selectively grown at the inner luminal layer of the tube similar to physiological ureter by using a bio-chamber under a dynamic conditions. The relative expression values for smooth muscle genes (smoothelin and calcium binding proteins-S100A4) and urothelial cells (cytokeratin 18 and 5, uroplakin) genes were upregulated within 2 weeks of culture.

The inventive apparatus and methods disclosed herein provide simple, rapid, and ease to form molded tissue scaffolds with complex designs that can be seeded with stem cells for creating biologically and mechanically functional tissues/grafts for organs such as in the urinary tract, as well as other applications, such as in the intestines, and vascular applications.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

“At least” a certain value is understood as that value or more. For example, “at least 10,” is understood as “10 or more”; “at least 20” is understood as “20 or more.” As used herein, “less than” a specific value is understood to mean that value and less. For example “less than 10” is understood to mean “10 or less.”

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value.

### EXAMPLES

### Cell Culture

Human urothelial cells (hUCs), human smooth muscle cells (hSMCs) and human adipose derived mesenchymal stem cells (hADSCs) were purchased from Sciencell (Carlsbad, Calif.). Experiments were performed with all cell types between passage 3 and 4. Smooth muscle cells were cultured in SMC medium consisting of a basal SMC medium (Sciencell, CA) with SMC growth supplement, 10% FBS and 1% Penn-Strep. hADSCs were cultured until P3 using growth media consisting of F-12/DMEM with L-Glutamine, 15 mM HEPES, 10% fetal bovine serum (FBS) and 1 ng/ml basic FGF (Life Technologies, Grand Island, N.Y.). At Passage 3, medium was changed to smooth muscle induction medium consisting of MCDB 131 medium (Sigma-Aldrich, St. Louis, Mo.) with 1% FBS and 100 U/ml of heparin (Sigma-Aldrich, MO) for over two weeks. hUCs were cultured on Poly (L-Lysine) (Sciencell, CA) coated cell culture flasks till P3 in a growth medium that consists of basal UC medium (Sciencell, CA) with UC growth supplement and 1% Penn-Strep (Life Technologies). Medium was changed every 2-3 days for all cell types.

### Engineering Molded Tubular Tissue Scaffolds

Collagen scaffolds were prepared by neutralizing a sterile bovine skin type I collagen (5 mg/mL in HCl, 10 mL) (Cosmo Bio, Tokyo, Japan) with 8.8 mL of Dulbecco's Modified Eagle's Medium (DMEM) with 1 g/L D-Glucose, L-glutamate, 110 mg/L Sodium Pyruvate (Life Technologies, NY), 1.0 mL of fetal bovine serum (FBS) and 0.2 mL of HEPES (×1, 1M) solution at 4° C. The freshly mixed solution was then injected into a balloon chamber with a sintered plastic mold (RKI Instruments rod-JJS Tech., IL) with either tubular or hollow shape that has a thin polycarbonate filter film (as an example—10 um mesh size) wrapped on it. After ˜1 h of incubation at 37° C. for fibrogenesis, collagen was further condensed by extracting water either under partial vacuum or against the contractile pressure of the balloon. The balloon chamber was opened up, and in some cases; the tube was vertically kept on a rotating plate in a humidity-chamber (39° C., 40% RH) for further drying the scaffold for 3 h. To create porous tubes, 1.5 gm of camphor microparticles (grinded and filtered through 250 μm Nylon filter) were added and suspended into the collagen solution that leaves behind pores on sublimation or dissolving and washing in ethanol. On rehydration, the tube was slowly pulled off from the mold. Prior to seeding cells on these scaffolds, tubes were treated with 0.2% peracetic acid and 4% ethanol (in PBS) for 6 h followed by three PBS washes for at least one hour each. For scaffolds with PRP and HA, DMEM was added with PRP lyophilized powder (49) (PRP-5 wt % or 10 wt % of total volume, HA-5% wt/wt) before neutralizing collagen solution. Similarly, for tubes that were embedded with cells, 0.5 million cells per cm length of the tubes were added to 1.0 mL of DMEM (taken out of the neutralizing stock solution) that was added to the freshly mixed collagen and neutralizing solution. Crosslinked tubes were prepared by shaking them in PBS solutions of THPP (0.01 M, Sigma-Aldrich, MO) for 2 h. Molded collagen scaffolds showed a typical fibrous structure and possessed a fibril-band like morphological structures.

To create molded porous tissue scaffold tubes, 2.0 g of camphor microparticles (grinded and filtered through 250 μm Nylon filter) were added and suspended into the collagen solution that leaves behind pores on sublimation. On rehydration, the tube was slowly pulled off from the mold. Prior to seeding cells on these scaffolds, tubes were treated with 0.2% peracetic acid and 4% ethanol (in PBS) for 6 h followed by three PBS washes for at least one hour. For scaffolds with PRP, DMEM was added with PRP lyophilized powder (5 wt % or 10 wt % of total volume) before neutralizing collagen solution. Similarly, for the tubes that are embedded with cells, 2 million cells per cm length of the tubes were added to 1 mL of DMEM (taken out of the neutralizing stock solution) that was added to the freshly mixed collagen and neutralizing solution. Crosslinked tubes were prepared by shaking them in PBS solutions of THPP (0.01 M, Sigma Aldrich) for 2 h.

### Cell Seeding of Molded Tubular Collagen Scaffolds

MSCs or SMCs seeded were allowed to attach scaffolds with or without embedded cells in 15 mL centrifuge tubes (Becton Dickinson, NJ) with their respective cell culture media for 6 h at 37° C. on a shaker (Corning, Tewksbury, Mass.). The cell seeding density was 2×10⁶/cm length of the tubes. UCs were seeded in the lumen of the seeded scaffolds after 1 week of growing SMCs in a following procedure: First, one end of the tubular scaffold was blocked with a customized polyimide stopper that was further fastened with a thin PTFE tape. Second, two million UCs/cm length of the tube was added to the lumen of the tubular scaffold; third, the other end of the tubular scaffold was blocked with a polyimide stopper and fastened with a thin PTFE tape. The seeded tubular scaffold was transferred to a 15 mL centrifuge tube that contained a mixture of SMCs and UCs culture media (50:50). For allowing cells to adhere to the lumen, the centrifuge tube was closed and kept flat but rolling on a shaker at 37° C. Cell culture medium was added into the centrifuge tube in a sufficient quantity to submerge the tubular scaffold in the flat position of the centrifuge tube. After 6 h of shaking, cell-seeded scaffold was transferred to a six-well cell culture plate and cultured overtime in a mixed cell culture medium (50:50 SMCs and UCs).

### PRP Isolation

Bovine whole blood was received and processed to collect PRP as previously reported (European J. Dentistry 4, 395-402 (2010)). Bovine blood was centrifuged in vacuum tubes at 16,000 RCF for 20 minutes, forming layers of platelets-poor plasma and blood cells. The platelet-poor plasma and the top 6 mm of the cell component phase were collected and centrifuged at 400×g for 15 minutes, creating a platelet-poor phase separated by a buffy coat from PRP. The buffy coat and PRP phase were collected and used in downstream experiments.

### Mechanical Testing

Mechanical properties of the tubular scaffolds were studied by determining the radial burst pressure strength and tensile stress-strain measurements in both longitudinal and transverse directions of the tube. Mean burst pressure strength of the tubular scaffolds were determined by an OMEGA DPG4000-1K digital manometer (OMEGA, Stamford, Conn.). One end of the tube was attached to a syringe pump (New Era Pump Systems, Farmingdale, N.Y.) connected to the manometer via a three-way adaptor, while the other end was tightly secured. Tubes were inflated with PBS solution through a syringe pump at 3.3 mL/min, the corresponding pressure values were recorded using OMEGA DPG4000-SW 1.12 software. Tensile properties of the collagen tubes in both longitudinal and transverse or circumferential directions were tested with a Bose tensile testing instrument (Enduratec ELF 3200, 225 N load cell) at a strain rate of 0.1 mm/s. Data were recorded using the WinTest software and processed using Microsoft Excel and Prism 6.0 software programs.

### Microstructure of the Scaffolds by SEM and TEM

SEM: Collagen tubes were fixed overnight at 4° C. in 2.5% glutaraldehyde in 100 mM sodium cacodylate buffer and 0.1% tannic acid (pH 7.2-7.4). Samples were washed in a 100 mM sodium cacodylate buffer with 3% sucrose and 3 mM MgCl₂. Samples were post-fixed in 0.8% potassium ferrocyanide and reduced with 1% osmium tetroxide for 1 h on ice, in the dark, followed by H₂O washes. Samples were dehydrated using a graded ethanol series before hexamethyldisilanaze (HMDS) rinses. After drying overnight in a desiccator, dry samples were mounted to Pelco SEM stubs using carbon tape and sputter coated with 10 nm of gold palladium and imaged on a LEO 1530 FESEM.

TEM: Collagen tube and trachea samples were fixed overnight at 4° C. in 2.5% glutaraldehyde in 100 mM sodium cacodylate buffer and 0.1% tannic acid (pH 7.2-7.4). Samples were washed in 100 mM sodium cacodylate buffer with 3% sucrose and 3 mM MgCl₂. Samples were post-fixed in 0.8% potassium ferrocyanide and reduced with 1% osmium tetroxide for 1 h on ice, in the dark, followed by distilled water rinses and En-bloc staining for 1 h at room temperature with 2% uranyl acetate (filtered). Samples were dehydrated using a graded ethanol series followed by two propylene oxide washes and then left overnight in a 1:1 ratio of propylene oxide and Eponate 12 (EPON). Samples were infiltrated with EPON resin and polymerized at 60° C. for 24 h. Ultra-thin sections (70-90 nm) of the samples were sliced using a Riechert Ultra-cut E ultramicrotome and placed on a coated copper 1×2 mm slot grids. Sections were stained first with 1% tannic acid (aqueous), 2% methanolic uranyl acetate and lead citrate before imaging on a Philips CM120 TEM operating at 80 kV. Images were digitally captured using an AMT XR-80 CCD camera (8 mega-pixel).

### Cell Viability, Morphology and Spatial Distribution Within Tubular Scaffolds

Cell-seeded scaffolds were incubated with cell culture medium containing 10% AlamarBlue® (Invitrogen, CA) for 4 h at 37° C. The fluorescence signal (540 nm excitation 590 nm emission) of a 100 μl medium aliquot for each sample was measured using a Synergy 2 microwell plate reader (BioTek, Winooski, Vt.). Culture medium without cells with 10% AlamarBlue was used as a negative control. Reduced fluorescence signals of the samples were evaluated according to the manufacturer's protocol at specified time points of days 7 and 14. Small thin cut pieces were assessed for cell viability using Live/Dead staining (Live/Dead® Viability/Cytotoxicity Kit for mammalian cells, Invitrogen) at different time points. The images were taken by a Zeiss microscope and processed in ImageJ. Cell morphology and distribution was studied by haematoxylin and eosin (HE) histostaining. Tubes were fixed with 4% formaldehyde and embedded in paraffin for overnight at 4° C. De-paraffinized 5-10 μm sections of tubes were stained with to reveal cell morphology, localization, and distribution throughout the tubes. Sections were imaged using an Olympus AX70 microscope.

### Gene Expression by qRT-PCR

The relative gene expression levels were assessed in a procedure as described below. Samples were harvested at different time points and snap frozen in liquid Nitrogen and stored at −80 ° C. Total RNA was isolated using the RNeasy mini kit (Qiagen, CA). Following extraction, 300 ng of RNA was used to generate the first strand cDNA using Random hexamers and Ready-To-Go You-Prime First-Strand Beads (GE healthcare, PA). The cDNA was subjected to a quantitative RT-PCR using gene specific primers and probe TaqManR Universal Mix II, No UNG (Thermofisher, MA) in a total volume of 20 μL per reaction and were run in triplicates in a 96 well plate along with the house keeping gene β-actin as controls in triplicates. RT-PCR experiment was performed using comparative CT Applied Biosystems at PCR cycle condition as follows: Hold at 50° C. for 2 min and at 95° C. for 10 min followed by 40 cycles of denaturing at 95° C. for 15 s and annealing at 60° C. for 1 min (for each cycle). A delta delta CT method was used to analyze the data points. The following genes were assessed using TaqMan's qRT-PCR primers/probe set (Table 2): smooth muscle: smoothelin (SMTH), SP100A4, collagen I alpha1, elastin, myocardin (MYCOD), calponin1 (CNN1); urothelial cells: cytokeratin 5 (KRT5), cytokeratin 18 (KRT18), uroplakin (UPA3A) and laminin (LAMA) and were normalized to β-Actin.

Morphological assessment of cell-seeded scaffolds was performed by a following procedure. In brief, the paraffin embedded cartilage samples were cut into 5 μm thick sections and placed onto a glass microscope slide after keeping for a few seconds in a 40° C. water bath. The glass slides with these sections were kept overnight at 40° C. on a hot plate. To rehydrate the samples, sections were rinsed sequentially, first with xylene followed by 100%, 95%, 80% ethanol, and deionized H₂O. Slides were then processed for antigen retrieval by keeping them in a target retrieval solution (Dako, CA) by steaming it for 45 minutes, followed by bringing it to the room temperature. After 10 min, slides were blocked in serum-free protein blocking buffer (Dako code X0909) for 1 h at 37° C. in dark. Slides were incubated with primary antibody (alpha-smooth muscle actin and pan cytokeratin, Abcams) at 4° C. overnight in a dark chamber followed by a rinse in a Dako wash buffer for 5 min. Secondary antibody in 1:800 dilution was added to the slides and kept at room temperature for 2 h, washed again with the Dako wash buffer for 5 min. Cell nuclei were stained with DAPI. After staining, samples were dried by washing with deionized H₂O, 80%, 95%, 100% ethanol and xylene. Samples on a cover slip were mounted with a Permount mounting solution and dried for 24 h. Samples were embedded in either paraffin wax or Tissue Embedding Media (Thermo Scientific, Logan, Utah) after freezing in liquid nitrogen, and sectioned with a Cryo-mill (Leica). Samples were imaged with Zeiss Discovery V2 dissection imaging microscope.

### Example 1

### Tissue Scaffold Molding: Seamless Engineered Scaffolds Made of Collagen

Specifically, we developed molded tissue scaffolds by injecting a freshly mixed ice-cold acid-solubilized collagen and its neutralizing aqueous solution into a mold chamber having a flexible outer wall (thin rubber balloon) (FIGS. 5A & B). Critical to this process are the support of a thin flexible wall, e.g. rubber balloon that holds the collagen fibrogenesis solution under fluid-pressure and the gravity in the mold chamber, and the shape-providing porous sacrificial or detachable internal mold element, which assists in expediting water extraction, further condensing the collagen solution on fibrogenesis under controlled vacuum or negative pressure.

### Example 2

### Modulating Mechanical and Biochemical Properties

Tissue scaffold molding results in tubular as well as partial or full bladder-like concave or hollow structures for neo urinary diversion conduit and bladder applications (FIGS. 5C & D) with user controlled mechanical properties and biochemical functionalities. Tubes with various wall thicknesses corresponding to the initial volume load (FIG. 5E) can be crosslinked to modulate tensile modulus and strength, and added with other biological agents, polymers and cells to impart unique biological and compositional features along with the specific structural scaffold designs. For example, we created molded tubular conduits from collagen doped with platelet-rich plasma (PRP) that has several growth factors to promote vascularization (Biomaterials 28, 4268-4276 (2007)), stem cells migration and recruitment (Polymer 58, 1-8 (2015)) and stimulate remodeling and healing the process (The Am. J. Sports Med 37, 2259-2272 (2009)); and molecules, such as hyaluronic acids (HA), without much compromising its overall mechanical properties while enhancing its biological modalities. Collagen tubes with 5-10% PRP maintained the mechanical properties (FIG. 5F) that can be further tuned by crosslinking collagen either by a simple thermal dehydration method-vitrification30 or by treating with external crosslinking agents, such as THPP31 (FIG. 5G). THPP cross-linking enhanced the Young's modulus and breaking stress although with a compromised % elongation value (<50-60%) (FIG. 5G).

We further compared the mechanical performance of the molded tubes (FIGS. 5H&I), in terms of tensile modulus and radial burst pressure strength values to PLGA electrospun tubes (Tengion™) that degrade overtime in PBS losing its strength and modulus by 10 folds within 14 days. The molded tubes surpassed the radial burst strength of the physiological ureter in human adults with a radial burst strength of ˜150-200 mmHg and a volume expansion of 2.3 fold per 100 mmHg pressure increase without any leakage or rupture (FIG. 5I). By appropriate wall thickness, crosslinking and biological factors, the tubes can further be modulated for its user-controlled biomechanical properties. We, further demonstrate the versatility of the process by designing and developing a wide spectrum of shapes and structures with tunable physical biophysical properties.

### Example 3

### Designer Scaffolds with Unprecedented Flexibilities

Molded tubular scaffolds that capture some design aspects of the native ureters were created. The versatility of collagen molding further enabled us to create scaffolds with a plethora of design possibilities in both longitudinal and cross-sectional planes, including structural diversion or tubular manifold (FIGS. 6A,B). It was also possible to create small diameter guiding channels to facilitate neural growth and blood supplies within the wall of the tubular scaffolds. We further developed tubes with diameter size varying from 300-500 μm to 1 cm and a full length of 7-8 cm (FIG. 6C). Tubes with micro-size diameters can be specifically developed for rodent urinary studies. Another challenges with the existing collagen conduits are the collapsed cylindrical inner periphery and possible kinks on bending the conduit, which is a possibility when implanted inside the body during the natural movement. Furthermore, material properties-stretchability and porosity are critical to their biomechanical functions, i.e. volume expansion under urodynamic pressure, and cellular functions in terms of migration and proliferation. Therefore, we created porous and stretchable tubes (FIGS. 6D-F).

### Example 4

### In Vitro Stem Cell Culture & Differentiation

MSCs seeded on collagen tubular scaffolds with no external crosslinking agents or process, are viable and proliferating throughout the scaffold after 72 h of seeding (FIG. 6A). Similarly, crosslinked tubular scaffolds via vitrification supported hADSCs growth overtime as shown by the live-dead staining (FIG. 6B) (PLGA as a control). In contrast to collagen scaffolds, number of cells (quantified by an Alamar blue staining, FIG. 6C) decreased overtime on PLGA scaffolds, possibly due to degradation of PLGA scaffold and local increase in pH. SEM images showed layer-by-layer arrangement of dense collagen fibers in hydrothermally cross-linked tubular scaffolds, where cells grew mostly on top of the surface (FIG. 6D).

To further facilitate cell penetrations and increase the overall surface area reachable to cells, porous collagen tubular scaffolds were designed by adding porogens (FIG. 6E). SEM showed porous structures of the tubes across the cross-sectional plane. Cells proliferated on the peripheral surface and spread across the pores of the tubes although more profoundly in PRP-containing scaffolds possibly due to the less collagen density (FIG. 6E).

In another methods, we successfully embedded the cells while condensing collagen and created cell impregnated tube under a partial vacuum that is viable even after 7 days of culture (FIG. 6I). Confocal images of embedded hMSCs showed spatial arrangement of the cells across the center of the tube. H&E staining shows the elongated morphology of the cells at the outer periphery, while more round cell morphology toward inner periphery (FIG. 6J). Seeded urothelial cells after 7 days of embedded hSMCs culture, proliferated at the peripheral-surface of the tube that can be selectively grown at the inner luminal layer of the tube similar to physiological ureter by using a bio-chamber under a dynamic conditions. The relative expression values for smooth muscle genes (smoothelin and calcium binding proteins-S100A4) and urothelial cells (cytokeratin 18 and 5, uroplakin) genes were upregulated within 2 weeks of culture.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A mold apparatus for making a molded tissue scaffold comprising an inlet/outlet adaptor, wherein said inlet/outlet adaptor comprises an inlet port and an outlet port which can allow fluids and gases to pass through the inlet or outlet port of the inlet/outlet adaptor, said inlet/outlet adaptor further comprising an internal mold element comprised of a sintered material which is semi-permeable or porous and said internal mold element defining a hollow interior space which connects to the outlet port of the inlet/outlet adaptor and communicates with the outlet port of the inlet/outlet adaptor, said internal mold element is capable of allowing gas and fluid to pass through the exterior of the internal mold element into the hollow interior space of the internal mold element and exit out of the outlet port of the inlet/outlet adaptor; the mold apparatus further comprises a mold chamber which is comprised of at least one wall comprising a flexible material which defines the inside and outside of the mold chamber, and encloses the internal mold element, and which is fastened at one end, to the inlet/outlet adaptor; the inlet port of the at least first adaptor communicates with the interior of the mold chamber such that fluid and a liquid tissue composition can enter into the mold chamber and be contained within said chamber; the liquid tissue composition can be added to the chamber via the inlet port at sufficient pressure to expand the flexible wall of the mold chamber such that the wall of the mold chamber will provide counter pressure to the liquid in the mold chamber and press against the internal mold element.

2. The mold apparatus of claim 1, wherein the apparatus further comprises at the end opposite of the inlet/outlet adaptor, a plug or impermeable wall, or a second adaptor, or nothing in case of hollow bladder or tubular scaffold that would use only balloon whose mouth is fastened to only one mold.

3. The mold apparatus of claim 1, wherein the internal mold element is bulbar or concave and comprises only a single inlet/outlet adaptor.

4. The mold apparatus of claim 1, wherein the flexible wall of the mold chamber has a spherical or balloon shape attached to the inlet/outlet adaptor.

5. The mold apparatus of claim 1, wherein the internal mold element is solid and the mold chamber communicates with one or more inlets of the one or more adaptors.

6. The mold apparatus of claim 1, wherein the inlet/outlet adaptor can be made from any rigid durable materials such as stainless steel, plastic, or glass.

7. The mold apparatus of claim 1, wherein the flexible wall of the mold chamber can be formed from any rubbery material or stretchable material-such as natural rubber or EPDM rubber etc.

8. The mold apparatus of claim 1, wherein the flexible wall of the mold chamber is translucent or permeable to wavelengths of light which can allow initiation of cross-linking of the tissue scaffold solution, such as UV or infrared wavelengths of light.

9. A method for making a molded tissue scaffold comprising the steps of:
a) obtaining a solution comprising one or more fibrous proteins suitable for use as a tissue scaffold;
b) combining the solution of a) with at least a second solution which will promote fibrogenesis and vitrification of the protein solution of a);
c) adding the combined solution of b) into the inlet of a mold apparatus capable of containing the solution of b) under pressure and gravity, and which comprises an internal mold element which is semi-permeable and communicates at one end to the outlet of the mold apparatus;
d) condensing the solution of b) in the expandable mold chamber of the mold apparatus of c; until the scaffold has desirable thickness and sufficient tensile strength; and
e) removal of the molded tissue scaffold from the mold.

10. The method of claim 9, wherein the method further comprises the step of: f) further mechanical or biological tuning or processing of the vitrified tissue scaffold.

11. The method of claim 9, wherein the fibrous protein of a) is collagen.

12. The method of claim 9, wherein the fibrous protein solution of a) is solubilized via acidification of the protein solution.

13. The method of claim 9, wherein the at least one second solution is a solution comprising a neutralizing buffer solution.

14. The method of claim 9, wherein the at least one second solution is a solution comprising a cross-linking agent.

15. The method of claim 9, wherein other polymers, cells, extracellular matrix components can be mixed in the second solution.

16. The method of claim 9, wherein the at least one second solution comprises a porogen.

17. The method of claim 16 wherein the porogen is selected from the group consisting of camphor particles, menthol, effervescents, and ammonium carbonate.

18. The method of claim 9, wherein the method further comprises the addition of at least one active agent in the solution of a).

19. The method of claim 18, wherein the at least one active agent is a drug, or growth factor, polymers, biopolymers, decellularized tissue particles, and florescent markers.

20. The method of claim 9, wherein the method further comprises the addition of at least one or more mammalian cells.

21. The method of claim 20, wherein the mammalian cells are stem cells.

22. The method of claim 9, wherein the molded tissue scaffold is in the shape of an organ of the body.

23. The method of claim 22, wherein the molded tissue scaffold is in the shape selected from the group consisting of: a ureter, bladder, urethra, small intestine, and a blood vessel.

24. A molded tissue scaffold comprising one or more fibrous proteins having the 3-dimensional shape of an organ of the body.

25. The molded tissue scaffold of claim 24, wherein the one or more fibrous proteins is collagen.

26. The molded tissue scaffold of claim 24, wherein the one or more fibrous proteins are cross-linked.

27. The molded tissue scaffold of claim 24, wherein the molded tissue scaffold optionally comprises biopolymers, cells, extracellular matrix components.

28. The molded tissue scaffold of claim 24, further comprising at least one active agent.

29. The molded tissue scaffold of claim 28, wherein the at least one active agent is a drug, or growth factor, polymers, biopolymers, decellularized tissue particles, and florescent markers.

30. The molded tissue scaffold of claim 24, further comprising at least one or more mammalian cells.

31. The molded tissue scaffold of claim 28, wherein the at least one or more mammalian cells are stem cells.

32. The molded tissue scaffold of claim 24, wherein the molded tissue scaffold is in a shape selected from the group consisting of: a ureter, bladder, urethra, small intestine, and a blood vessel.

33. The molded tissue scaffold of claim 28 for use in replacement of an organ in a body of a subject in need thereof.

34. The molded tissue scaffold of claim 28 for use in replacement of an organ which is diseased or non-functional.

35. The molded tissue scaffold of claim 28 for use in the augmentation or supplementation of an organ in a body of a subject in need thereof.
